(19) United States
(12) Patent Application Publication
(10) Pub. No.: US 2021/0236095 A1
(43) Pub. Date: Aug. 5, 2021

(54) ULTRASOUND CT IMAGE RECONSTRUCTION METHOD AND SYSTEM BASED ON RAY THEORY

(71) Applicant: HUAZHONG UNIVERSITY OF SCIENCE AND TECHNOLOGY, Hubei (CN)

(72) Inventors: Ming YUCHI, Hubei (CN); Mingyue DING, Hubei (CN); Xiaoyue FANG, Hubei (CN); Yun WU, Hubei (CN); Junjie SONG, Hubei (CN); Liang ZHOU, Hubei (CN); Qiude ZHANG, Hubei (CN); Kuolin LIU, Hubei (CN)

(73) Assignee: HUAZHONG UNIVERSITY OF SCIENCE AND TECHNOLOGY, Hubei (CN)

(21) Appl. No.: 16/968,151

(22) PCT Filed: Apr. 28, 2019
(86) PCT No.: PCT/CN2019/084712

§ 371 Date: Aug. 6, 2020

(30) Foreign Application Priority Data
Apr. 11, 2019 (CN) 201910286906.7

(51) Int. Cl. A61B 8/08; A61B 8/15

(52) U.S. Cl. CPC A61B 8/5207; A61B 8/08; A61B 8/15

(57) ABSTRACT

The disclosure is related to the technical field of functional imaging, and discloses an ultrasound CT image reconstruction method and system based on ray theory, wherein the method includes an ultrasound CT sound speed reconstruction method and an ultrasound CT attenuation coefficient reconstruction method based on ray theory; the ultrasound CT sound speed reconstruction method based on ray theory includes: (1) extraction of the difference in travel time; (2) calculate the ray path that the acoustic wave passes from the transmitting array element to the receiving array element; (3) solution for inverse problem: by using the quasi-Newton method to solve the path-slowness-time equation system, the speed reconstruction value vector of the object to be measured can be obtained.

### BACKGROUND

### Technical Field

The disclosure relates to a transmission ultrasound imaging mode in ultrasound tomography, belonging to the technical field of functional imaging, and more particularly, to an ultrasound CT image reconstruction method and system based on ray theory.

### Description of Related Art

Ultrasound CT refers to the use of ultrasound probes to transmit ultrasound wave to objects and receive reflection data or transmission data, and the data is used to reconstruct ultrasound tomographic images in order to observe three-dimensional information inside the object. Ultrasound inspection has the advantages of low price and harmless to human body. With the rapid development of probe processing technology and computer's high-performance computing capabilities, ultrasound tomography technology has once again become a popular topic for researchers in recent years.

Ultrasound CT imaging can be classified into two imaging modes, reflection imaging and transmission imaging. For collecting reflection information from multiple directions, the reflection image of ultrasound CT has a higher image resolution in order to assist the doctor to see small lesion tissue. Through transmission data, functional parameters such as speed of sound and attenuation coefficient can be reconstructed, which belongs to the field of functional images. Some studies have shown that in the early stage of the lesion, the lesion tissue changes in functional parameters first and then structural changes follow. Therefore, transmission imaging is of great significance for the early imaging and diagnosis of lesions, and the lesions can be diagnosed earlier.

The reconstruction method for ultrasound CT sound speed is basically the same as the reconstruction method for attenuation coefficient. Taking the reconstruction method for ultrasound CT sound speed as an example, the reconstruction method for ultrasound CT sound speed includes a reconstruction method based on ray theory and a reconstruction method based on wave theory. The reconstruction method based on the wave theory has higher imaging resolution, but is easily affected by small error disturbances; therefore such a method is not stable enough, and the amount of calculation is extremely large, which is not suitable for actual application. The reconstruction method model based on the ray theory is simpler with higher robustness and less amount of calculation. At present, the reconstruction method model based on the ray theory is an efficient, stable, and practical reconstruction method for sound speed.

Currently, few studies are conducted on reconstruction methods for ultrasound CT sound speed in China and foreign countries. The main reason is that there are some technical difficulties: it is difficult to build an ultrasound CT system and acquire data; the reconstruction method for ultrasound CT sound speed involves a large-scale of matrix operation and requires a lot of computational costs; there is an inverse problem to be solved in the reconstruction process of ultrasound CT sound speed, and therefore it is an issue to solve the inverse problem.

There are also some difficulties in the reconstruction method for ultrasound CT sound speed based on ray theory: it is considerably difficult to accurately extract a travel time, and is more easily affected by noise and system error; there are too many calculation methods for ray path and the methods are applied differently in different circumstances; there is also an inverse problem to be solved in the method, and therefore it is an issue to solve the inverse problem.

SUMMARY

Technical Problem

In view of the above defects or the needs for improvement of the related art, the purpose of the disclosure is to provide an ultrasound CT image reconstruction method and system based on ray theory, by improving the overall process of the method, especially through the optimization of ray theory, and the use of specific ray calculating and processing method, it is possible to realize fast and stable ultrasound CT sound speed reconstruction and ultrasound CT attenuation coefficient reconstruction, thereby realizing the ultrasound CT image reconstruction based on the ray theory.

In order to achieve the above purpose, according to an aspect of the disclosure, an ultrasound CT sound speed reconstruction method based on ray theory is provided, which is characterized by including the following steps:

(1) Extraction of the difference in travel time:

First, based on the same transmitting array element and receiving array element, the ultrasound transmission wave data from pure water and the object to be measured are collected respectively after the ultrasound wave is transmitted, and the respective corresponding pure water data and the data of the object to be measured are obtained respectively according to the channels.

Then use the AIC method to extract the travel time of pure water data and the data is recorded as tof_{water}; then, determine the matching window, take tof_{water} as the starting point, and take the time t_{water_max} at the maximum amplitude of pure water data as the end of window; the window length is recorded as w, and the window data in the matching window is recorded as W_{water}.

Then look for the sliding window whose window length is maintained at w on the to-be-measured object data in the corresponding channel. The window data in this sliding window is recorded as W_{object}; then, W_{object} and W_{water} are correlated to each other so as to calculate the cross-correlation coefficient; adjust the starting point of the sliding window, thereby sliding the sliding window to obtain a series of cross-correlation coefficients. Choose the sliding window corresponding to the cross-correlation coefficient with the largest value among these cross-correlation coefficients as the search result of the sliding window, and record the starting point of the search result of the sliding window as tof_{object}, then the difference in travel time is Δtof=to f_{object}−tof_{water}.

Next, adjust the channel and repeat the extraction process, and finally obtain a series of travel time difference Δtof corresponding to a series of channels.

(2) Calculate the ray path that the acoustic wave passes from the transmitting array element to the receiving array element.

Record the total number of a series of effective travel time differences Δtof obtained in step (1) as nₜ, and the imaging area is divided so that the grid number of the imaging area satisfies Σ×Σ, wherein Σ is a positive integer. When \sqrt{nₜ} is an integer, Σ is equal to \sqrt{nₜ}. When \sqrt{nₜ} is a non-integer, Σ is an integer obtained by ceiling, flooring, or rounding off \sqrt{nₜ}.

The imaging area is established in the two-dimensional plane rectangular array element coordinate system corresponding to the transmitting array element and the receiving array element. Then, for each group of transmitting array element-receiving array element, a straight line is connected between the coordinate position of the transmitting array element in the array element coordinate system and the coordinate position of the receiving array element in the array element coordinate system, thereby obtaining the path length of the connection line in each grid in the imaging area, and then obtaining the two-dimensional Σ×Σ matrix associated with the path length of each grid. Thereafter, the matrix is vectorized to obtain a vector about the path length in each grid. Finally, the vectors regarding the path length in each grid obtained by each group of transmitting array element-receiving array element are arranged to form a two-dimensional Σ²×Σ² matrix path matrix L regarding all transmitting array element-receiving array element groups.

(3) Solution for inverse problem:

Vectorize the effective travel time difference Δtof obtained in step (1) to obtain ΔT; then construct the path-slowness-time equation system as shown in equation (3):

LΔS=ΔT (3)

Specifically, ΔS is the amount of change in slowness to be solved; then, the quasi-Newton method is used to solve the equation system, and a one-dimensional vector ΔS containing Σ² elements is obtained. Thereafter, the slowness of ultrasound wave in water is added to the amount of change in slowness ΔS, take the reciprocal, and the speed reconstruction value vector of the object to be measured can be obtained.

According to another aspect of the disclosure, the disclosure provides a reconstruction method for ultrasound CT attenuation coefficient based on ray theory, characterized in that the method includes the following steps:

First, based on the same transmitting array element and receiving array element, the energy parameters from pure water and the object to be measured are collected respectively after the ultrasound wave is transmitted, and the respective corresponding pure water energy parameters and the energy parameter of the object to be measured are obtained respectively according to the channels. Then the ratio of the energy parameter of the object to be measured to the energy parameter of pure water is calculated.

Next, adjust the channel, repeat the extraction and calculation process, and finally obtain the energy parameter ratio corresponding to a series of channels.

(2) Calculate the ray path that the acoustic wave passes from the transmitting array element to the receiving array element.

Record the total number of a series of energy parameter ratios obtained in step (1) as nₜ, and the imaging area is divided so that the grid number of the imaging area satisfies Σ×Σ, wherein Σ is a positive integer. When \sqrt{nₜ} is an integer, Σ is equal to \sqrt{nₜ}. When \sqrt{nₜ} is a non-integer, Σ is an integer obtained by ceiling, flooring, or rounding off \sqrt{nₜ}.

The imaging area is established in the two-dimensional plane rectangular array element coordinate system corresponding to the transmitting array element and the receiving array element. Then, for each group of transmitting array element-receiving array element, a straight line is connected between the coordinate position of the transmitting array element in the array element coordinate system and the coordinate position of the receiving array element in the array element coordinate system, thereby obtaining the path length of the connection line in each grid in the imaging area, and then obtaining the two-dimensional Σ×Σ matrix associated with the path length of each grid. Thereafter, the matrix is vectorized to obtain a vector about the path length in each grid. Finally, the vectors regarding the path length in each grid obtained by each group of transmitting array element-receiving array element are arranged to form a two-dimensional Σ²×Σ² matrix path matrix L regarding all transmitting array element-receiving array element groups.

(3) Solution for inverse problem:

Vectorize the series of energy parameter ratios obtained in step (1) to obtain ΔP; then construct the path-attenuation-energy parameter equation system as shown in equation (4):

LΔA=ΔP (4)

Specifically, ΔA is the amount of change in attenuation coefficient to be solved; then, the quasi-Newton method is used to solve the equation system, and a one-dimensional vector ΔA containing Σ² elements is obtained. Thereafter, the attenuation coefficient of ultrasound wave in water is added to the amount of change in attenuation coefficient ΔA, and the attenuation coefficient reconstruction value vector of the object to be measured can be obtained.

According to still another aspect of the disclosure, the disclosure provides an ultrasound CT image reconstruction method using the above-described ultrasound CT sound speed reconstruction method based on ray theory, characterized in that the method utilizes the ultrasound CT sound speed reconstruction method based on ray theory as described in claim 1, and further includes the following steps:

(4) Imaging: Two-dimensionalize the obtained speed reconstruction value vector of the object to be measured to form Σ×Σ matrix; then obtain a two-dimensional pixel image based on the Σ×Σ matrix, and each pixel in the two-dimensional pixel image corresponds to the sound speed value.

As a preferred embodiment of the disclosure, the two-dimensional pixel image is obtained by performing logarithmic compression, grayscale mapping on the sound speed value, and finally displayed.

According to yet another aspect of the disclosure, the disclosure provides an ultrasound CT image reconstruction method using the ultrasound CT attenuation coefficient reconstruction method based on the ray theory as described in claim 2, and the method is characterized in that the method utilizes the ultrasound CT attenuation coefficient reconstruction method based on ray theory as claimed in claim 2 further includes the following steps:

(4) Imaging: Two-dimensionalize the obtained attenuation coefficient reconstruction value vector of the object to be measured to form Σ×Σ matrix; then obtain a two-dimensional pixel image based on the Σ×Σ matrix, and each pixel in the two-dimensional pixel image corresponds to the attenuation coefficient value.

As a preferred embodiment of the disclosure, in the step (4), the two-dimensional pixel image is obtained by performing logarithmic compression, grayscale mapping on the attenuation coefficient value, and finally displayed.

According to still another aspect of the disclosure, the disclosure provides an ultrasound CT sound speed reconstruction system based on ray theory, characterized in that the system includes:

A travel time difference extraction module, configured to: based on the same transmitting array element and receiving array element, collect ultrasound transmission wave data from pure water and the object to be measured respectively after transmitting ultrasound wave, and obtain respective corresponding pure water data and data of objects to be measured according to channels; utilize an AIC method to extract the travel time of the pure water data and then record the travel time as tof_{water}; determine a matching window and record the starting point as tof_{water}; record the time t_{water_max} at maximum amplitude of pure water data as the end of window, then the window length is recorded as w, and the window data in the matching window is recorded as W_{water}; find the sliding window whose window length is maintained at w on the data of the object to be measured in the corresponding channel. The window data in the sliding window is recorded as W_{object}; W_{object} and W_{water} are correlated to each other to calculate the cross-correlation coefficient; the starting point of the sliding window is adjusted to slide the sliding window to obtain a series of cross-correlation coefficients. Choose the sliding window corresponding to the cross-correlation coefficient with the largest value among these cross-correlation coefficients as the search result of the sliding window, and record the starting point of the search result of the sliding window as tof_{object}, then the difference in travel time is Δtof=tof_{object}−tof_{water}. Adjust the channel, repeat the extraction process, and finally obtain a series of travel time differences Δtof corresponding to a series of channels.

A calculation module for calculating the ray path that the acoustic waves pass from the transmitting array element to the receiving array element, configured to: record the total number of a series of effective travel time differences Δtof as nₜ, and the imaging area is divided so that the grid number of the imaging area satisfies Σ×Σ, wherein Σ is a positive integer. When \sqrt{nₜ} is an integer, Σ is equal to \sqrt{nₜ}. When \sqrt{nₜ} is a non-integer, Σ is an integer obtained by ceiling, flooring, or rounding off \sqrt{nₜ}. The imaging area is established in the two-dimensional plane rectangular array element coordinate system corresponding to the transmitting array element and the receiving array element. For each group of transmitting array element-receiving array element, a straight line is connected between the coordinate position of the transmitting array element in the array element coordinate system and the coordinate position of the receiving array element in the array element coordinate system, thereby obtaining the path length of the connection line in each grid in the imaging area, and then obtaining the two-dimensional Σ×Σ matrix associated with the path length of each grid. The matrix is vectorized to obtain a vector about the path length in each grid. The vectors regarding the path length in each grid obtained by each group of transmitting array element-receiving array element are arranged to form a two-dimensional Σ²×Σ² matrix path matrix L regarding all transmitting array element-receiving array element groups.

An inverse problem solution module, configured to vectorize the obtained effective travel time difference Δtof to obtain ΔT; then construct the path-slowness-time equation system as shown in equation (3):

LΔS=ΔT (3)

Specifically, ΔS is the amount of change in slowness to be solved.

The quasi-Newton method is used to solve the equation system, and a one-dimensional vector ΔS containing E² elements is obtained. Thereafter, the slowness of ultrasound wave in water is added to the amount of change in slowness ΔS, take the reciprocal, and the speed reconstruction value vector of the object to be measured can be obtained.

According to the last aspect of the disclosure, the disclosure provides an ultrasound CT attenuation coefficient reconstruction system based on ray theory, characterized in that the system includes:

An energy parameter extraction module, configured to, based on the same transmitting array element and receiving array element, the energy parameters from pure water and the object to be measured are collected respectively after the ultrasound wave is transmitted, and the respective corresponding pure water energy parameters and the energy parameter of the object to be measured are obtained respectively according to the channels. Then the ratio of the energy parameter of the object to be measured to the energy parameter of pure water is calculated. Adjust the channel, repeat the extraction and calculation process, and finally obtain the energy parameter ratio corresponding to a series of channels.

A calculation module for calculating the ray path that the acoustic waves pass from the transmitting array element to the receiving array element, configured to: record the total number of a series of energy parameter ratios as nₜ, and the imaging area is divided so that the grid number of the imaging area satisfies Σ×Σ, wherein Σ is a positive integer. When \sqrt{nₜ} is an integer, Σ is equal to \sqrt{nₜ}. When \sqrt{nₜ} is a non-integer, Σ is an integer obtained by ceiling, flooring, or rounding off \sqrt{nₜ}. The imaging area is established in the two-dimensional plane rectangular array element coordinate system corresponding to the transmitting array element and the receiving array element. For each group of transmitting array element-receiving array element, a straight line is connected between the coordinate position of the transmitting array element in the array element coordinate system and the coordinate position of the receiving array element in the array element coordinate system, thereby obtaining the path length of the connection line in each grid in the imaging area, and then obtaining the two-dimensional Σ×Σ matrix associated with the path length of each grid. The matrix is vectorized to obtain a vector about the path length in each grid. The vectors regarding the path length in each grid obtained by each group of transmitting array element-receiving array element are arranged to form a two-dimensional Σ²×Σ² matrix path matrix L regarding all transmitting array element-receiving array element groups.

An inverse problem solution module, configured to vectorize the obtained series of energy parameter ratios to obtain ΔP; then construct the path-attenuation-energy parameter equation system as shown in equation (4):

LΔA=ΔP (4)

Specifically, ΔA is the amount of change in attenuation coefficient to be solved.

Then, the quasi-Newton method is used to solve the equation system, and a one-dimensional vector ΔA containing Σ² elements is obtained. Thereafter, the attenuation coefficient of ultrasound wave in water is added to the amount of change in attenuation coefficient ΔA, and the attenuation coefficient reconstruction value vector of the object to be measured can be obtained.

Through the above technical solutions conceived by the disclosure, compared with the related art, since the technical solution of the disclosure is based on the ray theory and adopts parameters such as the travel time difference (Δtof) or energy parameter ratios in the reconstruction process, it is possible to achieve fast and stable ultrasound CT sound speed reconstruction and ultrasound CT attenuation coefficient reconstruction. On basis of the so-called ray theory, it is assumed that the propagation medium is relatively uniform, and the propagation path of ultrasound in a homogeneous medium can be approximated as rays.

Taking the sound speed reconstruction method based on ray theory as an example, the main steps include extraction of travel time, calculation of ray path, and solution of inverse problem:

First of all, the travel time difference (Δtof) is extracted from the data. The travel time is the time when the waveform reaches the receiving position. This is the starting time of the first waveform in the received waveform. The travel time difference refers to the difference in the travel time between phantom data and pure water data. The disclosure combines the cross-correlation (CC) method and the mutual information method (AIC), and introduces the maximum amplitude position information (MAX) to extract the travel time difference Δtof between the phantom data and the pure water data, which is called AIC-MAX-CC method in abbreviation.

Then the ray path that the acoustic wave passes from the transmitting array element to the receiving array element is calculated. In the disclosure, on the premise that the propagation medium is relatively uniform, so the path is approximated by a straight path. Then, the focus of the problem is shifted to calculating the acquisition of an intersection point of two connection lines passing through the grid. After acquiring the intersection point, the distance between every two intersection points is calculated in sequence, then the length of the path in a single grid can be obtained.

Finally, the path-slowness-time equation system is established and solved, that is, the establishment and solution of inverse problems. Slowness is the reciprocal of the sound speed. The slowness in the disclosure refers to the slowness increment of the phantom relative to pure water, the path is the length that the propagation path crosses the grid, and the time is the travel time difference between the phantom data and the pure water data. The disclosure adopts the quasi-Newton method to solve the equation system. The quasi-Newton method is a method for solving an equation system through repeated calculations, which is between the gradient descent method and the Newton method, and is a good method for optimizing repeated calculations.

In addition to using the energy parameter ratios to replace the travel time difference (Δtof), and using the path-attenuation-energy parameter equation system to replace the path-slowness-time equation system, the attenuation coefficient reconstruction method based on ray theory is basically similar to the sound speed reconstruction method based on ray theory.

In summary, the advantages of the sound speed reconstruction method and system based on the ray theory provide by the disclosure are as follows: 1. the travel time difference, rather than the travel time itself, is used in the construction of the equation system, which reduces the impact caused by the system error; 2. the extraction of the travel time difference adopts the cross-correlation method in combination with the mutual information method, and introduces the maximum amplitude position information, referred to as AIC-MAX-CC method in short, which has strong anti-noise ability; 3. the calculation of path is simple, based on the assumption that the propagation medium of sound speed is uniform, the path calculation problem is shifted to the problem of calculating the intersection point of two connection lines passing through the grid, which is simple and effective; 4. the quasi-Newton method is adopted to solve the inverse problem, which avoids the calculation of the Hessian matrix, the calculation amount is small, and the accuracy of solution is high. The advantages of the attenuation coefficient reconstruction method and system based on the ray theory are as follows: 1. the construction of the equation system adopts the energy parameter ratios rather than the energy itself, which reduces the impact caused by system errors; 2. the calculation of path is simple, based on the assumption that the propagation medium of sound speed is uniform, the path calculation problem is shifted to the problem of calculating the intersection point of two connection lines passing through the grid, which is simple and effective; 3. the quasi-Newton method is adopted to solve the inverse problem, which avoids the calculation of the Hessian matrix, the calculation amount is small, and the accuracy of solution is high.

### BRIEF DESCRIPTION OF THE DRAWINGS

FIG. 1 is a schematic diagram of AIC method.

FIG. 2 is a schematic diagram of AIC-MAX-CC method, wherein the upper diagram corresponds to pure water data and the lower diagram corresponds to phantom data.

FIG. 3 is a schematic diagram of a straight path.

FIG. 4 is an ultrasound CT ring array (i.e., UCT ring array) and a breast custom phantom 1768-00 (CIRSINC, USA).

FIG. 5 is a reflection image reconstructed from a certain layer of data obtained by ultrasound CT scanning of the phantom.

FIG. 6 is the result of reconstructing the layer of data by the sound speed reconstruction algorithm provided by the disclosure (i.e., the sound speed image reconstructed by the algorithm of the disclosure).

FIG. 7 is an attenuation coefficient image obtained through reconstruction by a sound speed reconstruction algorithm in Embodiment 2 of the disclosure.

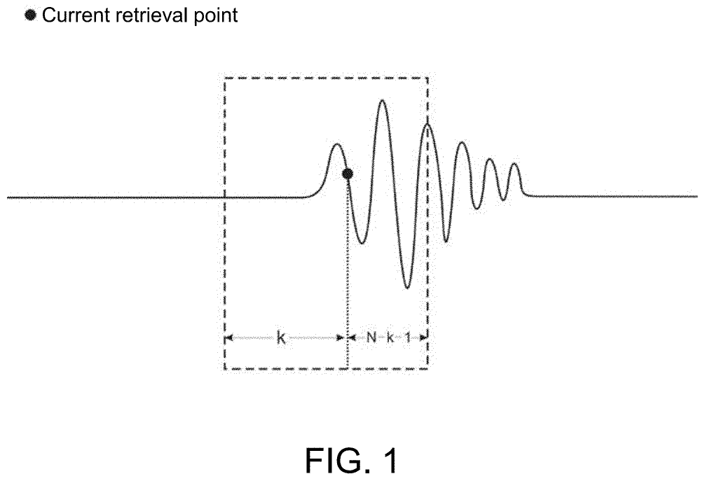

### DESCRIPTION OF THE EMBODIMENTS

In order to make the purpose, technical solutions and advantages of the disclosure clearer, the disclosure will be further described in detail in combination with the accompanying drawings and embodiments. It should be understood that the specific embodiments described herein are only used to explain the disclosure, and are not intended to limit the disclosure. In addition, the technical features involved in the various embodiments of the disclosure described below can be combined with each other as long as there is no conflict with each other.

### Embodiment 1

The sound speed reconstruction method based on the ray theory in the disclosure is to properly process the transmission data collected by the ultrasound CT system and finally make a reconstruction to obtain the ultrasound sound speed image. The steps include extracting the travel time, calculating the ray path, resolving the inverse problem and displaying the sound speed images.

The collected data is derived from the ultrasound CT hardware system. The hardware system mainly includes an ultrasound probe, a transmitting circuit module, a receiving circuit module, a data acquisition module, and a computer system. The connection between them and the way of they work with each other can be directly derived from existing technologies, such as literature (Junjie Song, S. W. L. Z., A Prototype System for Ultrasound Computer Tomography with Ring Array, in International conference Biomedical Image and signal processing. 2017). The hardware modules such as the transmitting circuit module, the receiving circuit module, and the data acquisition module can be constructed by referring to the existing technology. The ultrasound probe may be a ring array probe, a linear array probe, an area array probe, etc. Taking the ring array probe as an example, the transmission data is collected by using the ring transmission mode. The transmission wave is mainly received by the array element opposite to the position of the transmitting array element. Therefore, the disclosure uses the data received by the array element opposite to the transmitting array element to reconstruct the sound speed. If other linear array probes and area array probes are used, they can be processed in a similar manner.

First, the AIC method is adopted to extract the travel time of pure water data, which is recorded as tof_{water}.

The algorithm of the AIC method operates as follows: first, select a suitable window near the estimated travel time on the data, and the window length is N. Take the current retrieval point as the dividing point, divide the window into two segments, calculate the AIC value of the current retrieval point, and search the points in the window in sequence, and record the point with the smallest AIC value as the travel time point.

Specifically, reference for the initially estimated travel time point can be derived from the conventional operation in the related art. For example, the approximate travel time point can be calculated based on the theoretical sound speed of water, or the approximate location of travel time point can be observed by naked eyes on the waveform. The window length N is also selected based on the actual shape of the waveform and experiences, it can be increased or decreased as needed if the selection result does not meet the need. Reference for the calculation method of the AIC value can be directly derived from the related art.

Then determine the matching window, take tof_{water} as the starting point, and take the time t_{water_max} at the maximum amplitude of pure water data as the end of window; the window length is recorded as w, and the window data in the matching window is recorded as W_{water}.

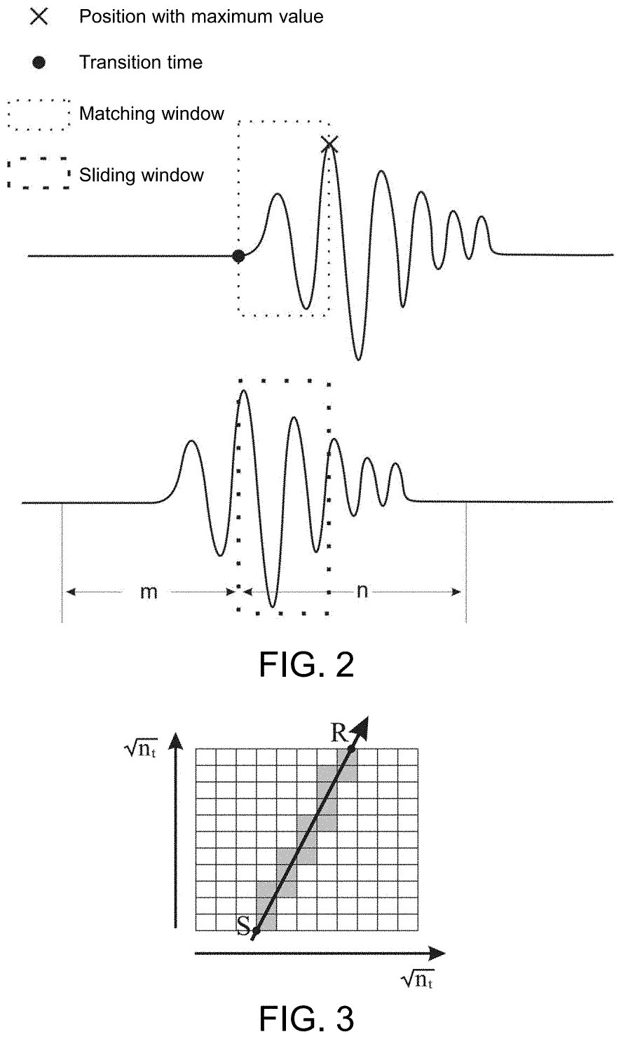

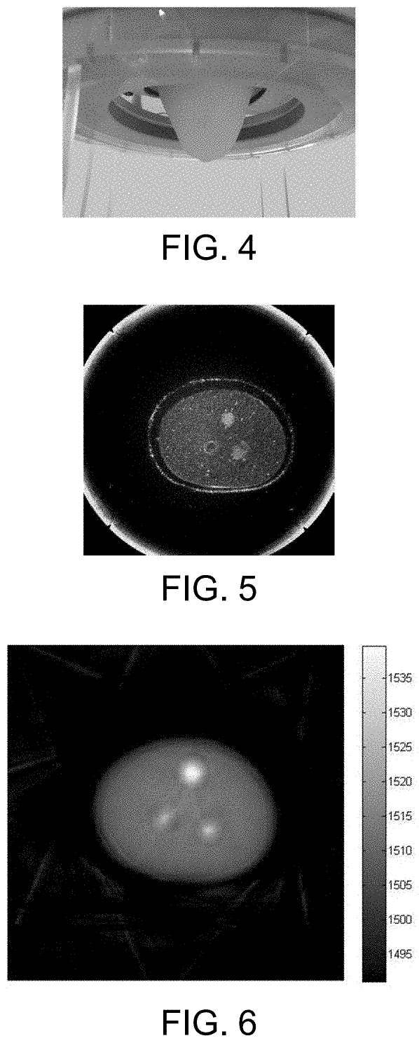

Then look for the sliding window with window length w on the phantom data of the corresponding channel, and the data of the sliding window is recorded as W_{object}. W_{object} and W_{water} are correlated to calculate the cross-correlation coefficient, and the sliding point with the largest cross-correlation coefficient is selected and recorded as p (p is negative when the starting point of sliding window is before tof_{water}, p is positive when the starting point of sliding window is after tof_{water}), then Δtof=p, as shown in FIG. 2. That is to say, the window data length of the sliding window is maintained as w, and then the sliding is performed. When sliding one point, one correlation coefficient is calculated, and the sliding point corresponding to the largest correlation coefficient is selected and recorded as p.

Since there are multiple channels, the travel time difference of each channel needs to be solved separately.

The imaging area is then divided. The size of the grid needs to be determined according to the number of effective travel time differences, that is, the number of effective equations of the path-slowness-time equation system. Invalid travel time difference such as some missing channel signals is an invalid one.

In order to maintain the positive definiteness of the equation, the number of unknown numbers and the number of equations need to maintain consistent. After extracting the travel time difference, it is necessary to remove the wrong channel. Assuming that the number of valid travel time differences after removal is nₜ, then the grid number is \sqrt{nₜ}*\sqrt{nₜ}. If \sqrt{nₜ} is a decimal, it can be rounded (any rounding rule such as ceiling, flooring or rounding off can be adopted).

Then calculate the ray path that the acoustic wave passes from the transmitting array element to the receiving array element. In the disclosure, on the premise that the propagation medium is relatively uniform, so the propagation path of sound wave is approximated by a straight line. Then, the focus of the problem is shifted to calculating an intersection point of two connection lines passing through the grid between two array element positions. After acquiring the intersection point, the distance between every two intersection points is calculated in sequence, and the distance is the length of the path in a single grid (if there is only one intersection point in a grid, the path length in the grid is 0). As shown in FIG. 3, it is a schematic diagram of a straight path, S is the transmitting array element, and R is the receiving array element. The \sqrt{nₜ}*\sqrt{nₜ} is vectorized (that is, the two-dimensional matrix \sqrt{nₜ}*\sqrt{nₜ} is turned into a one-dimensional column vector). After vectorization, the blank grid is zero, representing the grid is not crossed by the grid, the gray grid is a non-zero value. The non-zero value is the path length in the network, which means that the grid is crossed by the path. Then the size of the path matrix L of nₜ paths is nₜ×nₜ.

For the above grid, according to the dimensional parameters given by the probe manufacturer, the processing methods in the related art can be utilized to correspond each array element to a coordinate system, and the corresponding array element coordinates can be obtained.

After the path calculation is completed, the path-slowness-time equation system is constructed as shown in the following equation (3), wherein ΔS is the amount of change in slowness, ΔT is the travel time difference, and L is the nₜ×nₜ path matrix; the dimension of the equation (3) is the number of effective travel time difference.

LΔS=ΔT (3)

Use the quasi-Newton method to solve the equation system, and output ΔS (the obtained ΔS is a vector, which can be a positive value or negative value). The slowness of water plus this amount of change in slowness, take the reciprocal, then the speed reconstruction value of the phantom can be obtained. The obtained speed reconstruction value of the phantom is also in the form of vector.

Imaging step: Two-dimensionalize the speed reconstruction value of the phantom in the form of a vector (that is, the inverse process of the above \sqrt{nₜ}*\sqrt{nₜ} matrix vectorization) and then obtain a two-dimensional pixel image, each pixel in the two-dimensional pixel image is a sound speed value.

### Embodiment 2

Similar to Embodiment 1, the attenuation coefficient reconstruction method based on the ray theory in the disclosure includes:

The attenuation coefficient reconstruction method based on the ray theory in the disclosure is to properly process the transmission data collected by the ultrasound CT system, and finally make a reconstruction to obtain the ultrasound attenuation image. The steps include extracting signal energy parameters, calculating the ray path, solving the inverse problem, and displaying attenuation coefficient image.

The energy parameters may be the amplitude, intensity, and energy of the signal.

The calculation of the ray path is the same as in Embodiment 1.

The solution to the inverse problem is the same as in Embodiment 1.

After the path calculation is completed, the path-attenuation-energy parameter equation system is constructed, as shown in the following equation (4), wherein ΔA is the amount of change in the attenuation coefficient and ΔP is the energy parameter ratio, that is, the energy parameter ratio of the energy parameter of phantom data to the energy parameter of the water data.

LΔA=ΔP (4)

Adopt the quasi-Newton method to solve (4), then ΔA is obtained. The attenuation coefficient of water plus the amount of change in this attenuation coefficient, then the reconstruction value of the attenuation coefficient of the phantom can be obtained.

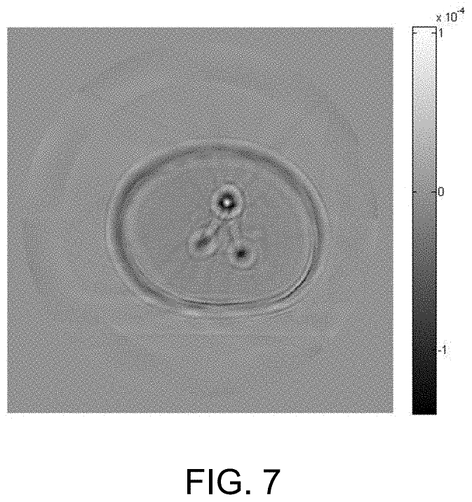

Finally, an image display of the attenuation coefficient is shown in FIG. 7.

In the above embodiment, for the obtained ultrasound image, logarithmic compression and grayscale mapping may be sequentially performed according to the method in the related art to obtain an ultrasound image with a grayscale value within a specified range.

The disclosure is applicable to various commercial ultrasound CT system probes, such as ring probes, linear array probes, area array probes, concave arrays, etc.

Those skilled in the art can easily understand that the above are only preferred embodiments of the disclosure and are not intended to limit the disclosure. Any modification, equivalent replacement and improvement made within the spirit and principle of the disclosure should fall within the scope of the disclosure.

1. An ultrasound CT image reconstruction method using an ultrasound CT sound speed reconstruction method based on ray theory, comprising the following steps:
(1) extracting a travel time difference:
first, based on the same transmitting array element and receiving array element, ultrasound transmission wave data from pure water and an object to be measured are collected respectively after an ultrasound wave is transmitted, and respective corresponding pure water data and the data of the object to be measured are obtained respectively according to channels;
then use an AIC method to extract a travel time of the pure water data and the data is recorded as tof_{water}; then, determine a matching window, take tof_{water} as a starting point, and take a time t_{water_max} at the maximum amplitude of the pure water data as the end of window; a window length is recorded as w, and a window data in the matching window is recorded as W_{water};
then look for a sliding window whose window length is maintained at w on the data of the object to be measured in the corresponding channel, the window data in the sliding window is recorded as W_{object}; then, W_{object} and W_{water} are correlated to each other so as to calculate a cross-correlation coefficient; adjust a starting point of the sliding window, thereby sliding the sliding window to obtain a series of cross-correlation coefficients, choose the sliding window corresponding to the cross-correlation coefficient with the largest value among the cross-correlation coefficients as a search result of the sliding window, and record the starting point of the search result of the sliding window as tof_{object}, then the difference in travel time is Δtof=tof_{object}−tof_{water};
next, adjust the channel and repeat the extraction process, and finally obtain a series of travel time difference Δtof corresponding to a series of channels;
(2) calculate a ray path that acoustic wave passes from the transmitting array element to the receiving array element:
record the total number of a series of effective travel time differences Δtof obtained in step (1) as nₜ, and an imaging area is divided so that a grid number of the imaging area satisfies Σ×Σ, wherein Σ is a positive integer, when \sqrt{nₜ} is an integer, is equal to \sqrt{nₜ}, when \sqrt{nₜ} is a non-integer, Σ is an integer obtained by ceiling, flooring, or rounding off \sqrt{nₜ};
the imaging area is established in a two-dimensional plane rectangular array element coordinate system corresponding to the transmitting array element and the receiving array element, then, for each group of transmitting array element-receiving array element, a straight line is connected between a coordinate position of the transmitting array element in an array element coordinate system and a coordinate position of the receiving array element in the array element coordinate system, thereby obtaining a path length of the connection line in each grid in the imaging area, and then obtaining a two-dimensional Σ×Σ matrix associated with the path length of each grid, thereafter, the matrix is vectorized to obtain a vector about the path length in each grid, finally, vectors regarding the path length in each grid obtained by the each group of transmitting array element-receiving array element are arranged to form a two-dimensional Σ²×Σ² matrix path matrix L regarding all of the transmitting array element-receiving array element groups;
(3) solution for inverse problem:
vectorize the effective travel time difference Δtof obtained in step (1) to obtain ΔT;
then construct a path-slowness-time equation system as shown in equation (3):
LΔS=ΔT (3)
wherein, ΔS is an amount of change in slowness to be solved; then, a quasi-Newton method is utilized to solve the equation system, and a one-dimensional vector ΔS containing Σ² elements is obtained, thereafter, slowness of ultrasound wave in water is added to the amount of change in slowness ΔS, take a reciprocal, and a speed reconstruction value vector of the object to be measured can be obtained.

2. An ultrasound CT image reconstruction method using an ultrasound CT attenuation coefficient reconstruction method based on ray theory, comprising the following steps:
(1) first, based on the same transmitting array element and receiving array element, energy parameters from pure water and the object to be measured are collected respectively after the ultrasound wave is transmitted, and the respective corresponding pure water energy parameters and the energy parameter of the object to be measured are obtained respectively according to the channels, then a ratio of the energy parameter of the object to be measured to the energy parameter of pure water is calculated;
next, adjust the channel, repeat the extraction and calculation process, and finally obtain an energy parameter ratio corresponding to a series of channels;
(2) calculate the ray path that the acoustic wave passes from the transmitting array element to the receiving array element;
record the total number of the series of energy parameter ratios obtained in step (1) as nₜ, and the imaging area is divided so that the grid number of the imaging area satisfies Σ×Σ, wherein Σ is a positive integer, when \sqrt{ₜ} is an integer, Σ is equal to \sqrt{nₜ}, when \sqrt{nₜ} is a non-integer, Σ is an integer obtained by ceiling, flooring, or rounding off \sqrt{nₜ};
the imaging area is established in the two-dimensional plane rectangular array element coordinate system corresponding to the transmitting array element and the receiving array element, then, for each group of transmitting array element-receiving array element, the straight line is connected between the coordinate position of the transmitting array element in the array element coordinate system and the coordinate position of the receiving array element in the array element coordinate system, thereby obtaining the path length of the connection line in each grid in the imaging area, and then obtaining the two-dimensional Σ×Σ matrix associated with the path length of each grid, thereafter, the matrix is vectorized to obtain the vector about the path length in each grid, finally, the vectors regarding the path length in each grid obtained by the each group of transmitting array element-receiving array element are arranged to form the two-dimensional Σ²×Σ² matrix path matrix L regarding all of the transmitting array element-receiving array element groups;
(3) solution for inverse problem:
vectorize the series of energy parameter ratios obtained in step (1) to obtain ΔP; then construct a path-attenuation-energy parameter equation system as shown in equation (4):
LΔA=ΔP (4)
wherein, ΔA is an amount of change in attenuation coefficient to be solved; then, the quasi-Newton method is used to solve the equation system, and a one-dimensional vector ΔA containing Σ² elements is obtained; thereafter, an attenuation coefficient of ultrasound wave in water is added to the amount of change in attenuation coefficient ΔA, and the attenuation coefficient reconstruction value vector of the object to be measured can be obtained.

3. The ultrasound CT image reconstruction method using the ultrasound CT sound speed reconstruction method based on ray theory according to claim 1, wherein the method utilizes the ultrasound CT sound speed reconstruction method based on ray theory according to claim 1 and further comprises the following steps:
(4) imaging: two-dimensionalize the obtained speed reconstruction value vector of the object to be measured to form a Σ×Σ matrix; then obtain a two-dimensional pixel image based on the Σ×Σ matrix, and each pixel in the two-dimensional pixel image corresponds to a sound speed value.

4. The ultrasound CT image reconstruction method using the ultrasound CT sound speed reconstruction method based on ray theory according to claim 3, wherein in the step (4), the two-dimensional pixel image is obtained by performing logarithmic compression, grayscale mapping on the sound speed value, and finally displayed.

5. The ultrasound CT image reconstruction method using the ultrasound CT attenuation coefficient reconstruction method based on ray theory according to claim 2, wherein the method utilizes the ultrasound CT attenuation coefficient reconstruction method based on ray theory according to claim 2 and further comprises the following steps:
(4) imaging: two-dimensionalize the obtained attenuation coefficient reconstruction value vector of the object to be measured to form a Σ×Σ matrix; then obtain a two-dimensional pixel image based on the Σ×Σ matrix, and each pixel in the two-dimensional pixel image corresponds to an attenuation coefficient value.

6. The ultrasound CT image reconstruction method using the ultrasound CT attenuation coefficient reconstruction method based on ray theory according to claim 5, characterized in that, in the step (4), the two-dimensional pixel image is obtained by performing logarithmic compression, grayscale mapping on the attenuation coefficient value, and finally displayed.

7. An ultrasound CT sound speed reconstruction system based on ray theory, wherein the system comprising:
a travel time difference extraction module, configured to: based on the same transmitting array element and receiving array element, collect ultrasound transmission wave data from pure water and an object to be measured respectively after transmitting an ultrasound wave, and obtain respective corresponding pure water data and data of objects to be measured according to channels; utilize an AIC method to extract a travel time of the pure water data and then record the travel time as tof_{water}; determine a matching window and record a starting point as tof_{water}, record a time t_{water_max} at the maximum amplitude of the pure water data as the end of window, then a window length is recorded as w, and a window data in the matching window is recorded as W_{water}; find a sliding window whose window length is maintained at w on the data of the object to be measured in the corresponding channel, the window data in the sliding window is recorded as W_{object}; W_{object} and W_{water} are correlated to each other to calculate a cross-correlation coefficient; a starting point of the sliding window is adjusted to slide the sliding window to obtain a series of cross-correlation coefficients, choose the sliding window corresponding to the cross-correlation coefficient with the largest value among the cross-correlation coefficients as a search result of the sliding window, and record the starting point of the search result of the sliding window as tof_{object}, then the difference in travel time is Δtof=tof_{object}−tof_{water}; adjust the channel, repeat the extraction process, and finally obtain a series of travel time differences Δtof corresponding to a series of channels;
a calculation module for calculating a ray path that acoustic waves pass from the transmitting array element to the receiving array element, configured to: record the total number of the series of effective travel time differences Δtof as nₜ, and an imaging area is divided so that the grid number of the imaging area satisfies Σ×Σ, wherein Σ is a positive integer, when \sqrt{nₜ} is an integer, Σ is equal to \sqrt{nₜ}; when \sqrt{nₜ} is a non-integer, Σ is an integer obtained by ceiling, flooring, or rounding off \sqrt{nₜ}, the imaging area is established in a two-dimensional plane rectangular array element coordinate system corresponding to the transmitting array element and the receiving array element, for each group of transmitting array element-receiving array element, a straight line is connected between a coordinate position of the transmitting array element in a array element coordinate system and a coordinate position of the receiving array element in the array element coordinate system, thereby obtaining a path length of the connection line in each grid in the imaging area, and then obtaining a two-dimensional Σ×Σ matrix associated with the path length of each grid, the matrix is vectorized to obtain a vector about the path length in each grid, vectors regarding the path length in each grid obtained by the each group of transmitting array element-receiving array element are arranged to form a two-dimensional Σ²×Σ² matrix path matrix L regarding all of the transmitting array element-receiving array element groups;
an inverse problem solution module, configured to vectorize the obtained effective travel time difference Δof to obtain ΔT; then construct a path-slowness-time equation system as shown in equation (3):
LΔS=ΔT (3)
wherein, ΔS is an amount of change in slowness to be solved;
a quasi-Newton method is used to solve the equation system, and a one-dimensional vector ΔS containing Σ2 elements is obtained; thereafter, the slowness of ultrasound wave in water is added to an amount of change in slowness ΔS, take a reciprocal, and a speed reconstruction value vector of the object to be measured can be obtained.

8. An ultrasound CT attenuation coefficient reconstruction system based on ray theory, wherein the system comprising:
an energy parameter extraction module, configured to, based on the same transmitting array element and receiving array element, energy parameters from pure water and the object to be measured are collected respectively after an ultrasound wave is transmitted, and the respective corresponding pure water energy parameters and the energy parameter of the object to be measured are obtained respectively according to channels, then a ratio of the energy parameter of the object to be measured to the energy parameter of pure water is calculated, adjust the channel, repeat the extraction and calculation process, and finally obtain an energy parameter ratio corresponding to a series of channels;
a calculation module for calculating a ray path that the acoustic waves pass from the transmitting array element to the receiving array element, configured to: record the total number of a series of energy parameter ratios as nₜ, and an imaging area is divided so that a grid number of the imaging area satisfies Σ×Σ, wherein Σ is a positive integer, when \sqrt{nₜ} is an integer, Σ is equal to \sqrt{nₜ}, when \sqrt{nₜ} is a non-integer, Σ is an integer obtained by ceiling, flooring, or rounding off \sqrt{nₜ}, the imaging area is established in a two-dimensional plane rectangular array element coordinate system corresponding to the transmitting array element and the receiving array element, for each group of transmitting array element-receiving array element, a straight line is connected between a coordinate position of the transmitting array element in an array element coordinate system and a coordinate position of the receiving array element in the array element coordinate system, thereby obtaining a path length of the connection line in each grid in the imaging area, and then obtaining a two-dimensional Σ×Σ matrix associated with the path length of each grid, the matrix is vectorized to obtain a vector about the path length in each grid, vectors regarding the path length in each grid obtained by the each group of transmitting array element-receiving array element are arranged to form a two-dimensional Σ²×Σ² matrix path matrix L regarding all of the transmitting array element-receiving array element groups;
an inverse problem solution module, configured to vectorize the obtained series of energy parameter ratios to obtain ΔP; then construct a path-attenuation-energy parameter equation system as shown in equation (4):
LΔA=ΔP (4)
wherein, ΔA is an amount of change in attenuation coefficient to be solved;
then, a quasi-Newton method is used to solve the equation system, and a one-dimensional vector ΔA containing Σ² elements is obtained, thereafter, the attenuation coefficient of ultrasound wave in water is added to the amount of change in attenuation coefficient ΔA, and an attenuation coefficient reconstruction value vector of the object to be measured can be obtained.
